(19) United States
(12) Patent Application Publication
(10) Pub. No.: US 2008/0319271 A1
(43) Pub. Date: Dec. 25, 2008

(54) PERSONALIZED HEALTH MANAGEMENT TOOL

(76) Inventors: Cynthia A. Barnowski, Chanhassen, MN (US); William R. Guptail, Minnetrista, MN (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/765,115

(22) Filed: Jun. 19, 2007

(51) Int. Cl. A61B 5/00

(52) U.S. Cl. 600/300

(57) ABSTRACT

A personalized health management tool provides personalized health management information to one or more patients or members of a health plan for use both by the patient and the patient's healthcare provider(s) to improve the quality and efficiency of interactions between the patient and the provider(s). The personalized health management tool includes information relating to a patient's medical conditions and may include integrated guidelines that provide guidance on management and treatment of co-morbidities of the patient. The tool further includes information relating to the self-management of the patient's condition(s), information relating to the medical history of a patient, and/or information relating to the recommended care and treatment required for the patient's condition(s). For example, the health management tool may include EBM guidelines regarding the medical condition(s), integrated guidelines for multiple condition management, and/or information relating to resources related to the patient's medical condition(s) that are available to the patient for assistance with healthcare, financial issues, and/or social issues. A method for providing personalized health management information to one or more patients or members of a health plan includes identifying patients or members having one or more specific medical conditions; gathering personalized health data for each identified patient or member; and providing each identified patient or member with a personalized health management tool including information on the specific medical conditions, self-management of the specific medical conditions, treatment of the medical conditions for use by the patient's healthcare providers, and the personalized health data gathered for the respective identified patient or member.

### FIELD OF THE INVENTION

The present invention relates to a personalized health management tool, such as a personalized health kit, and also to a method for providing and updating the personalized health management tool for one or more patients or members of a health plan.

### BACKGROUND OF THE INVENTION

A lack of adherence to evidence-based medicine (“EBM”) guidelines by health care providers and patients in the management of even the most common chronic conditions has been widely documented. Challenges that providers often face in practicing EBM include: not having good, relevant information accessible when they need it; the need to spend too much time with patients collecting basic information about their recent medical history; difficulty in maintaining patient compliance to EBM protocol; and trying to apply a “one size fits all” EBM solution to all patients, regardless of their unique characteristics or circumstances.

For example, providers may spend much time getting “caught up” with how a patient is currently doing and what the patient has done since the previous office visit. Also, patient medical data may be incomplete, inaccurate, or dated. Additionally, it may be a struggle to get patients to change from their usual behaviors or routines to healthier ones. Patients also may have a difficult time understanding and participating in self-managing their medical condition(s). In many cases, the cost of medical care and the lack of available tools may be a significant factor causing lack of patient adherence to or self-management of a healthcare plan. Patients also tend to underutilize non-physician health professionals.

For example, patients with diabetes often do not consistently check their blood sugar. In other examples, patients, particularly elderly patients, do not remember what medications they are taking and/or are unable to provide accurate information to the health care provider (e.g., they know they were prescribed a blue pill, but do not know why or what it is for; they had an x-ray, but they cannot explain to the physician what the x-ray was for). In many cases, the result is physician time spent investigating, making phone calls to other physicians or other care providers, obtaining reports and tests, etc. This is often made more difficult by regulations under the Health Insurance Portability and Accountability Act, or HIPAA.

There is a need for a system and method that provides a remedy for the challenges faced by physicians, referenced above, in practicing EBM by improving the effectiveness of the limited time that patients have with their physicians. There is a further need for a system and method that provides valuable information to the physician for reference during office visits, so that the time the physician spends with the patient will be more productive. There is a need to provide valuable patient-specific information to the physician through the patient to promote patient-physician interaction. Additionally, there is a need for a system and method for allowing the physician to have more time to uncover deeper, more complex issues, such as those that may cause future medical complications that can be avoided, when face-to-face with a patient. There is similarly a need for a system and method for effectively engaging a patient in their treatment plan and reducing the patient's likelihood of non-compliance with that plan. There is a need for a system and method that promotes the patient-physician relationship. There is a further need for a system and method that promotes the patient-physician-insurance company relationship. Additionally, there is a need for a system and method that effectively improves the quality of medical care and financial outcomes relating to medical care.

### SUMMARY OF THE INVENTION

The present invention relates to a personalized health management tool that may be provided, for example, in the form of a personalized health kit. The kit may include information relating to a patient's medical condition and information relating to the self-management of that condition. The kit further may include information relating to the medical history of a patient, information relating to the care for the patient's medical condition, and patient health management or monitoring tools. The kit may further include information relating to EBM guidelines regarding the medical condition or care for the medical condition, or, when the patient has more than one health-related condition, the kit may include integrated guidelines to better enable the patient and healthcare providers to manage the patient's multiple conditions. The kit may also include information relating to resources related to the patient's medical condition that are available to the patient for assistance with healthcare, financial issues, social issues, provider information, insurance information, emergency information, and any other desired information. The information relating to the medical history of a patient may be gathered from the patient, a physician or other care provider, insurance claim data or other insurance information, and/or any other relevant source. In some embodiments the patient's medical conditions may include diabetes and/or congestive heart failure.

The present invention, in another embodiment, is a method for providing a personalized health management tool to one or more patients or members of a health plan. The method includes identifying one or more patients or members of a health plan having one or more specific medical conditions, gathering personalized health data for each of the identified patients or members, and providing to each identified patient or member a personalized health management tool including information on the specific medical condition(s), information concerning care relating to the specific condition(s), and the personalized health data for the patient or member. The personalized health management tool also may be updated.

The information on the specific condition(s) may include background information relating to the condition(s), treatments for the condition(s), information about daily living with the condition(s), the member's medical history, EBM guidelines, and or information relating to resources related to the member's medical condition that are available to the member for assistance with healthcare, financial issues, and/or social issues. The personalized health data for each patient or member may be gathered from the member of the health plan, a physician or other care provider, the medical history of the member, insurance claim data or other insurance information, evidence-based medicine sources, and/or any other relevant source. Updates for the gathered information may be provided to the patient or member.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 illustrates a general method for providing personalized health management information.

FIG. 2 illustrates an exemplary detailed method for providing personalized health management information.

FIG. 3 is a perspective view of a personalized health management tool in the form of notebook-style health kit.

FIG. 4 is a close-up perspective view of the notebook-style health kit of FIG. 3.

FIG. 5 illustrates an exemplary method for providing a personalized health management tool to one or more patients or members of a health plan.

### DETAILED DESCRIPTION

The personalized health management tool and method of providing this tool described herein may be implemented to enhance the physician-patient relationship and physician-patient interaction. Similarly, the personalized health management tool may promote active engagement with the patient and further promote patient compliance with a healthcare plan. The personalized nature of the tool avoids a “one size fits all” approach to the practice of EBM and provides patient-specific information to a physician via the patient's tool.

The present invention may be implemented to provide personalized medical health information and tools to patients, physicians, and other care providers. Some of the information provided includes, but is not limited to, information relating to the medical condition or medical conditions for particular patients, background information relating to the medical condition(s), the medications and/or treatments for the medical condition(s), self-management of the medical condition(s), questions that may be appropriate for a patient to ask relating to the medical condition(s), resources for various types of assistance, etc. Some tools that may be provided include, but are not limited to, record keeping forms for monitoring a patient's medical condition(s) and/or activities, tools for monitoring medications, doctor visit checklists, etc.

The present invention may be used by members of the health/medical industry, and particularly may be used by patients, physicians or other care providers, and insurance companies. The present invention further may promote patient and physician adherence to EBM guidelines.

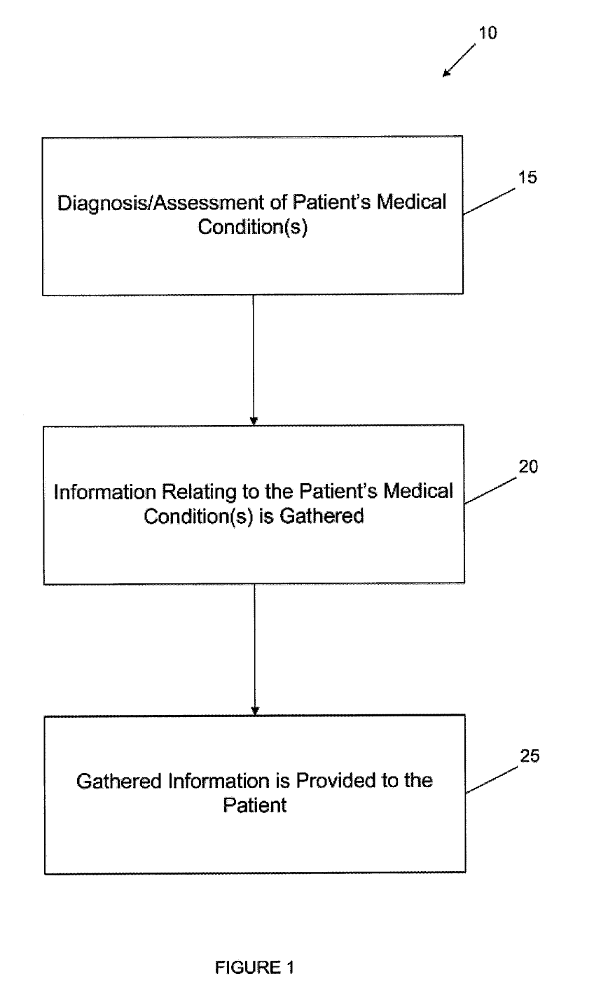

With reference to FIG. 1, a method 10 for providing a personalized health management tool includes patient diagnosis and/or assessment (15) to determine whether the patient has any existing medical conditions, characteristics indicative of existing medical conditions, characteristics or risk factors indicative of future medical conditions that may arise, etc., or any combination thereof. Patient, as used herein, includes patients, members of a healthcare plan, or any other person seeking medical attention or assistance. Medical condition, as used herein, is meant to include medical/health conditions, ailments, pains, aches, mental conditions, allergies, viruses and diseases, including chronic diseases and co-morbidity, any other condition that a physician or other care provider is typically sought for assistance, or any combination thereof.

In one embodiment of the present system and method, a patient may be diagnosed or assessed by a physician. In other embodiments, the patient may be diagnosed or assessed by other care providers, such as a nurse or personal care manager. For example, the diagnosis or assessment may be performed to determine whether the patient suffers from, or is a candidate for, diabetes, including Type I diabetes, Type II diabetes, gestational diabetes, or pre-diabetes.

In another example, the diagnosis or assessment may be performed to determine whether the patient suffers from, or is a candidate for, congestive heart failure. In yet another example, the diagnosis or assessment may be performed to determine whether the patient suffers from, or is a candidate for, the combination of diabetes and congestive heart failure. In alternative embodiments, the diagnosis or assessment may be performed to determine whether the patient suffers from any medical condition or combination of medical conditions.

Subsequent to assessment of the patient, information relating to the patient's medical condition or combination of medical conditions may be gathered (20). Information relating to a patient's medical condition(s) may include, but is not limited to, the patient's medical history, background information relating to the medical condition(s), treatments and medications for the medical condition(s), information relating to daily living with the medical condition, EBM guidelines, and information relating to resources related to the patient's medical condition that are available to the patient for assistance with healthcare, financial issues, and/or social issues. In further embodiments, any information that may be helpful to a patient for understanding, coping with, self managing, etc. with the medical condition(s) may be included as a portion of the information gathered. In one embodiment, the information gathered, or a portion thereof, is personalized for, or tailored to, the patient. The information relating to the patient's medical condition(s) may be gathered from any number of sources, including but not limited to, the patient, the patient's physician(s), other physicians, other care providers, the patient's medical history, insurance claim data or other insurance information, and EBM. A patient may provide information through office visits with a physician or other care provider, questionnaires, diagnosis, etc. A physician or care provider may provide information through office visits with a patient, diagnosis of the patient, education and experience, etc.

In situations in which a patient has multiple medical conditions, the information provided to the patient and the healthcare provider may be formulated to take into account the co-morbidities of the patient. For example, certain medications or procedures that are used to treat one condition may not be appropriate when the patient also has a second condition. In one embodiment, EBM guidelines for each of the patient's multiple health conditions may be combined or integrated into integrated cluster condition guidelines to provide both the patient and the patient's healthcare providers with guidelines to enable them to manage the patient's multiple conditions. Thus, the personalized medical condition information provided to the patient and the healthcare provider may better enable the patient to simultaneously manage the multiple medical conditions and better enable the provider to diagnose, treat and advise the patient.

After information relating to the patient's medical condition or combination of medical conditions is gathered, the information may be provided to the patient (25). As discussed in detail below, the information, in one embodiment, may be provided to the patient as a tool in the form of in one comprehensive kit. In a further embodiment, the information may be provided to the patient in one health kit, such as but not limited to, a folder, a notebook, a binder, a portfolio, etc. In yet a further embodiment, an electronic copy of the information may be provided to the patient. The patient may also be able to enter and store information electronically. In one embodiment, the electronic copy may be editable.

After the information relating to the patient's medical condition(s) is provided to the patient, any portion of the process may be repeated in any order. For example, subsequent to providing the patient with the information relating to the patient's medical condition(s), the patient may be rediagnosed or reassessed. Similarly, subsequent to providing the patient with the information relating to the patient's medical condition(s), further information relating to the patient's medical condition(s) may be gathered and/or provided to the patient. For example, updates for the information may be gathered and/or provided to the patient. In some embodiments, updates for the information may be gathered and/or provided to the patient at regular intervals, such as once every month, once every six months, once every year, or any other suitable interval of time. Alternatively or additionally, updates may be provided as new information is received, upon patient request, or at any time.

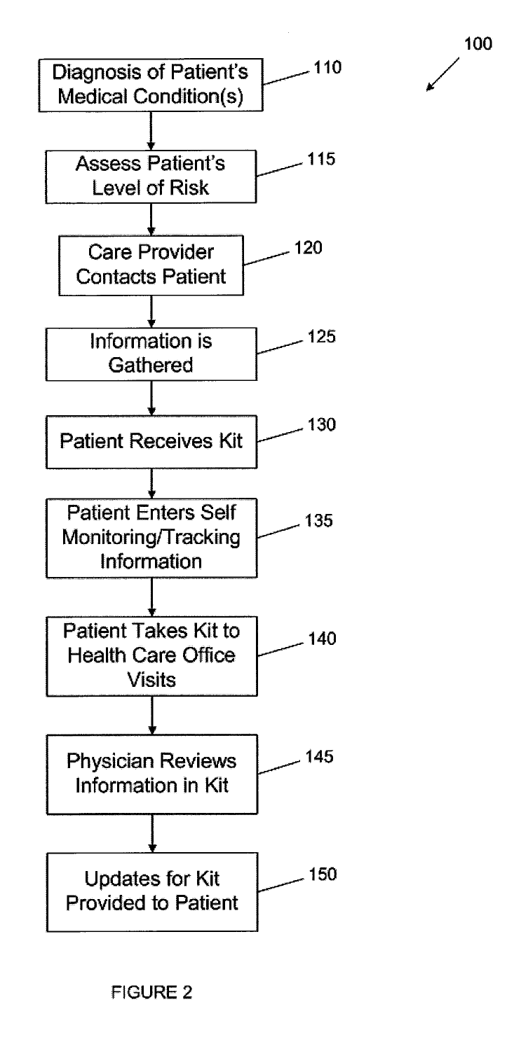

FIG. 2 illustrates an exemplary detailed method for providing a personalized management tool, generally referred to by the numeral 100. It should be understood that the flow chart in FIG. 2 is illustrative of one embodiment of the present invention and other embodiments exist without departing from the scope of the invention. Particularly, it is recognized that not all steps illustrated in FIG. 2 are required steps. Similarly, in some embodiments, more steps than those shown in FIG. 2 may be included.

In one embodiment of the present system and method, a patient (such as member of a health plan) may be diagnosed (110) to determine whether the patient has any existing medical conditions, characteristics indicative of existing medical conditions, characteristics or risk factors indicative of future medical conditions that may arise, etc., or any combination thereof. Similarly, the patient's level of risk may be assessed (115). In some embodiments, the level of risk may be used to determine whether a patient should be included in a particular health plan program, such as that described herein. Although diagnosis and assessment are illustrated in FIG. 2 as separate steps, it is contemplated that such diagnosis and assessment may be done as a single step. In one embodiment, a patient may be diagnosed or assessed by a physician. In other embodiments, the patient may be diagnosed or assessed by other care providers, such as a nurse or personal care manager. In yet other embodiments, the physician or care provider who performs diagnosis of the patient may be different than the physician or care provider who performs the assessment.

In one example, the diagnosis or assessment may be performed to determine whether the patient suffers from, or is a candidate for, diabetes, including Type I diabetes, Type II diabetes, gestational diabetes, or pre-diabetes. In another example, the diagnosis or assessment may be performed to determine whether the patient suffers from, or is a candidate for, congestive heart failure. In yet another example, the diagnosis or assessment may be performed to determine whether the patient suffers from, or is a candidate for, the combination of diabetes and congestive heart failure. In alternative embodiments, the diagnosis or assessment may be performed to determine whether the patient suffers from any medical condition or combination of medical conditions.

In some embodiments, after a patient has been diagnosed and/or assessed, the patient may be contacted by a care provider, such as but not limited to, a nurse or personal care manager. In other embodiments, the patient may be contacted by a physician that may or may not be the physician that diagnosed and/or assessed the patient. The care provider, in one embodiment, may provide information to the patient in the form of a personalized health management tool such as a notebook-style or other health kit. In further embodiments, the care provider may further interview the patient for additional information that may be included in the kit, lead to other information that may be included in the kit, or upon which further information that may be included in the kit is based.

Information relating to the patient's medical condition or combination of medical conditions, also may be gathered (125). Information relating to a patient's medical condition(s) may include, but is not limited to, the patient's medical history, background information relating to the medical condition(s), treatments and medications for the medical condition(s), information relating to daily living with the medical condition, EBM guidelines, and information relating to resources related to the patient's medical condition that are available to the patient for assistance with healthcare, financial issues, and/or social issues. In further embodiments, any information that may be helpful to a patient for understanding, coping with, self managing, etc. with the medical condition(s) may be included as a portion of the information gathered. In one embodiment, the information gathered is personalized for, or tailored to, the patient. The information relating to the patient's medical condition(s) may be gathered from any number of sources, including but not limited to, the patient, the patient's physician(s), other physicians, other care providers, the patient's medical history, insurance claim data or other insurance information, and EBM. Co-morbidity may also be addressed, for example, by providing to the patient and the patient's healthcare providers integrated cluster condition guidelines based upon an integration of EBM guidelines for each of the patient's diseases or conditions. Such integrated guidelines may enable the patient and the patient's healthcare providers to manage better the patient's multiple medical conditions. A patient may provide information through office visits with a physician or other care provider, questionnaires, diagnosis, etc. A physician or care provider may provide information through office visits with a patient, diagnosis of the patient, education and experience, etc.

As previously discussed, after the information relating to the patient's medical condition(s) is gathered, it may then be provided to the patient as a personalized health management tool, for example, in the form of a health kit (130). The kit may be provided as one comprehensive item or be made up of a plurality of separate items. For example, the information may be provided to the patient as one or more folders, notebooks, binders, portfolios, etc. Alternatively or additionally, an electronic copy of the information may be provided to the patient. In one embodiment, the electronic copy may be editable, and the patient may. The health kit is discussed in further detail below.

Self-management and/or self-monitoring tools may be provided with the health kit. These tools may be integrated into the health kit and/or provided separately from the health kit and may be used in conjunction with the health kit. Self-management/monitoring tools may enable a patient to understand and track changes and progress, daily habits, activities and exercises, medications taken, including what medications, amount taken, and when taken, food and liquid intake, body weight, blood pressure, blood sugar level, or other medical statistics, etc. In some embodiments, the self-management/monitoring tools may include, but are not limited to, worksheets for the patient to record blood sugar level at various times of the day, weight, times of exercise and heart rate during the exercise, the medications taken (e.g., the name of the medication, the name of the physician that prescribed the medication, when the medication is taken, how much medication is taken, etc.), and checklists for physician visits. In other embodiments, the self-management/monitoring tools provided may include any tools that may be helpful for the patient and/or physician or other care provider to monitor a patient's changes and progress.

The health kit may also include health insurance information for the patient, a list of the patient's health care providers names and associated information (e.g., contact and specialty information), emergency contact information, and any other desired information.

The health kit may be designed to encourage the patient to bring the health kit along to office visits with a physician or other care provider (140). The health kit may promote better communication and interaction between the patient and physician. As previously stated, the health kit may include several pieces of important information, including but not limited to, the patient's medical history, background information relating to the medical condition(s), treatments and medications for the medical condition(s), and EBM guidelines, that may be helpful for the physician or other care provider to quickly and accurately obtain information relating to the patient's medical condition(s). The information in the health kit is kept up-to-date by periodic updates provided to the patient. Thus, the physician or other care provider may not need to spend too much time getting “caught up” with how the patient is currently doing and what the patient has done since the previous office visit and the provider-patient visit may be conducted more efficiently and productively. In one embodiment, the health kit may include information detailing what the patient should bring to each office visit and/or what the patient should do before each office visit.

The health kit may be designed such that the physician may review the health kit for background information relating to the patient, the patient's recent medical history, and EBM guidelines relating to the patient's medical condition(s). In a further embodiment, the physician may review the self-management tools provided in the health kit to determine patient changes and progress. In yet other embodiments, the physician may review any portion of the health kit that the physician feels may provide assistance during the office visit.

In one embodiment, additional information relating to the patient's medical condition(s) may be gathered subsequent to providing the patient with the health kit. As such, this additional information may be provided to the patient as updates to the health kit. In one embodiment, updates may be based on subsequent patient insurance claim data. In another embodiment, updates may be provided after a patient visits with a care provider. In yet another embodiment, updates may be provided after a periodic check-in with a care provider or other healthcare administrator. In a further embodiment, updates may be based on the information provided by the patient to the care provider or healthcare administrator. In some embodiments, updates may be sent at regular time intervals, such as once every month, once every six months, once every year, or any other suitable interval of time. In other embodiments, updates may be sent at random time intervals or upon request by the patient, patient's family, physician or other care provider, insurance company, etc. In a further embodiment, the interval of time for sending updates may be determined based on the medical condition or combination of medical conditions from which the patient suffers.

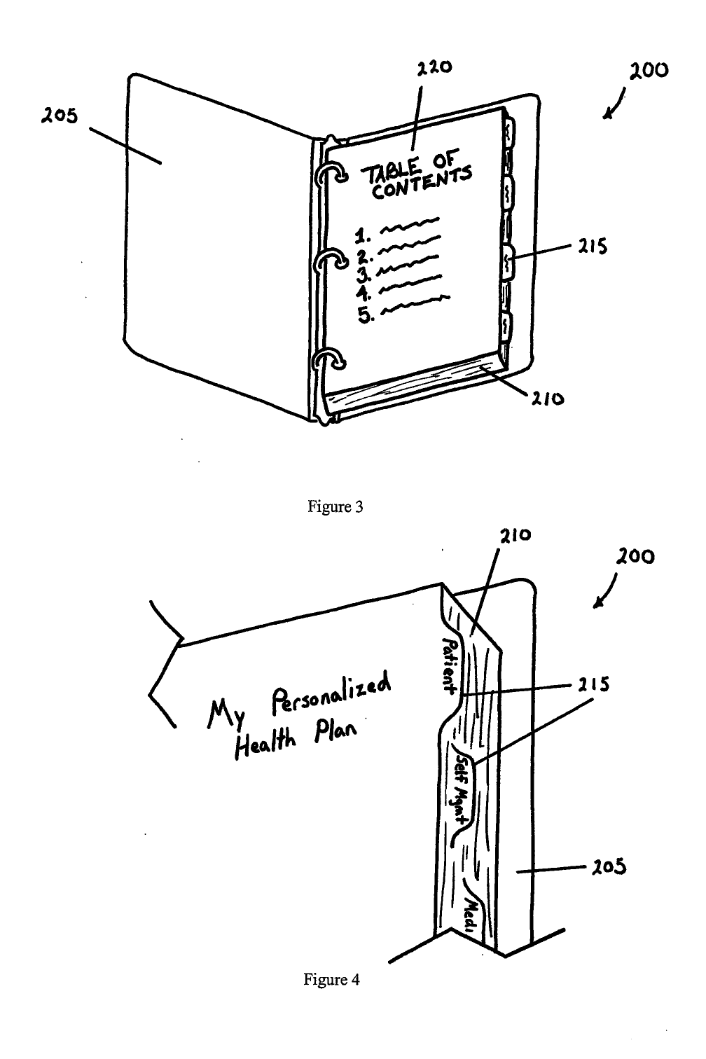

As illustrated in FIGS. 3 and 4, the information relating to the patient's medical condition(s) may be provided in one health kit 200, such as but not limited to, a folder, a notebook, a binder, a portfolio, etc. In one embodiment, the health kit 200 is a three-ring binder 205 having a plurality of pages containing the information relating to the patient's medical condition(s). Alternative formats, sizes, bindings, designs and media are also contemplated. Alternatively or additionally, an electronic copy of the information, such as a downloadable copy of the information, a copy on a compact disc, digital video disc, or other electronic media, etc., may be provided to the patient. In one embodiment, the electronic copy may be editable, and the patient may enter and store data. For example, the electronic copy may include self-management tools, as previously described, in which the patient may edit or add self-management information.

The health kit 200 may be a user-friendly source of information for both the patient and the physician. Furthermore, the health kit may provide one source of information for which the patient and physician need to refer. In one embodiment, the health kit 200 is divided into two main sections, a patient section and a physician section. In other embodiments, the health kit 200 is not separated into sections. In yet other embodiments, the health kit 200 is separated into more than two sections. For example, in some embodiments, there may be a care provider or nurse section. In some embodiments, there may be a family section that may be directed to or customized for the patient's family. Each section may include information relating to the patient's medical condition(s) that is tailored particularly for the person/people designated by that section. Similarly, each section may include words and language that are more easily understood by the target recipient(s). However, access need not be restricted to only the person/people designated for each section. For example, a patient may find assistance in the physician section. Similarly, the physician may find assistance in the patient section, such as the self-management tools.

In one embodiment, the health kit 200 may contain information relating to the patient's medical condition or combination of medical conditions. Information relating to a patient's medical condition(s) may include, but is not limited to, the patient's medical history, background information relating to the medical condition(s), treatments and medications for the medical condition(s), information relating to daily living with the medical condition, EBM guidelines, and information relating to available resources where the patient can look for healthcare assistance, financial assistance, and assistance with social issues. This information may include resources that are public or private and that are local, national, or worldwide. In further embodiments, any information that may be helpful to a patient for understanding, coping with, self managing, etc. with the medical condition(s) may be included as a portion of the information. For example, in one embodiment, the health kit 200 may include information relating to a patient's healthcare coverage. As previously mentioned, the health kit 200 may also include self-management tools that enable a patient to understand and track changes and progress, daily habits, activities or exercises, medications taken, including what medications, amount taken, and when taken, food and liquid intake, weight, blood pressure, blood sugar level, or other medical statistics, etc. In some embodiments, background information relating to the medical condition(s), information relating to daily living with the medical condition(s), and the self-management tools may be provided in a patient section of the health kit 200. In a further embodiment, the patient's medical history, information relating to treatments and medications for the medical condition(s) and EBM guidelines may be provided in a physician section of the health kit 200. In alternative embodiments, any of the information may be included in either section or in other sections of the health kit 200. In some embodiments, duplicate information may be provided in more than one section.

In a further embodiment, the health kit 200 may include tabs 215 for quick reference and access to particular information. For example, tabs 215 may be included that indicate the beginning of a patient section or a physician section. Other embodiments may include tabs 215 for background information, medical history, EBM guidelines, self-management tools, etc. In some embodiments, the tabs 215 may be color-coded for each section of the health kit 200. In other embodiments, the pages 210 upon which the information is printed may be color-coded for each section and/or each type/kind of information detailed by those pages 210. In yet other embodiments, other distinguishing features may be used to provide a manner of quick reference to particular portions or sections of the health kit 200. In further embodiments, the health kit may provide a table of contents 220.

In one embodiment, medical history information for a patient may be provided by the patient, the patient's physician(s), insurance claim data or other insurance information, or any other suitable source where medical history for a patient may be sought, such as, but not limited to, the patient's family members. A patient or a patient's family member may provide information through office visits with a physician or other care provider, questionnaires, etc. In some embodiments, one or more insurance companies may provide medical history data including, but not limited to, past medical and prescription claims data, summaries of patient physician visits, emergency room visits or other hospitalizations, and prescribed medications data.

In a further embodiment, medical history information for a patient may be provided by a comprehensive system such as that discussed in detail in co-pending U.S. patent application Ser. No. 11/559,148, filed Nov. 13, 2006, entitled “Integrated Electronic Healthcare,” herein incorporated by reference. The comprehensive system of U.S. patent application Ser. No. 11/559,148 generally relates to an integrated healthcare management system that provides a dynamic and comprehensive source of medical records and other health-related information.

As previously discussed, in one embodiment, the medical history information for a patient may be provided in a health kit 200. In a further embodiment, the medical history information may be provided in the physician section of a health kit 200. However, it is contemplated that the medical history may be located at any suitable point in a health kit 200.

Furthermore, the medical history information for a patient may be monitored throughout care for the patient. Updates for a health kit 200 relating to a patient's medical history may be sent to the patient at regular or non-regular time intervals, as requested by the patient, or at any time. In some embodiments, updates may be sent after changes in a patient's medical history have occurred. In other embodiments, updates may be sent at periodic time intervals, such as once every month, once every six months, once every year, or any other suitable time interval.

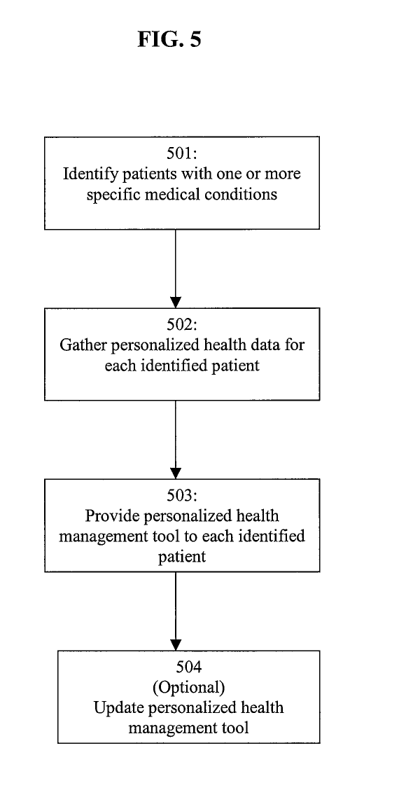

FIG. 5 illustrates a method for providing a personalized health management tool, such as the health kit described in detail above, in which one or more patients or members of a health plan having one or more specific medical conditions are identified (501); personalized health information (data) is gathered for the identified patients or members (502); and a personalized health management tool is provided to each identified patient or member (503). The personalized health management tool includes information on the specific medical condition(s) of the patient or member, information on care relating to the specific condition(s), and the personalized health information gathered for the patient or member. The information in the tool optionally may be updated (504), for example, periodically or as new information on the patient or specific condition(s) is received.

Although the present invention has been described with reference to preferred embodiments, persons skilled in the art will recognize that changes may be made in form and detail without departing from the spirit and scope of the invention.

We claim:

1. A personalized health management tool, comprising:
information relating to at least one medical condition of a patient;
information relating to self-management of the at least one medical condition; and
medical history information for the patient.

2. The personalized health management tool of claim 1, wherein the information relating to the at least one medical condition of a patient comprises evidence-based medicine guidelines.

3. The personalized health management tool of claim 1, further including information relating to the care for the at least one medical condition for the patient.

4. The personalized health management tool of claim 1, wherein the information relating to at least one medical condition of a patient comprises integrated guidelines relating to more than one condition of the patient.

5. The personalized health management tool of claim 1, further including information relating to available resources on healthcare assistance, financial assistance, or social issues assistance.

6. The personalized health management tool of claim 1, further including health insurance information for the patient.

7. The personalized health management tool of claim 1, further including health care provider information for the patient.

8. The personalized health management tool of claim 1, wherein the information relating to the medical history for the patient comprises information gathered from at least one of the patient, the physician or other care provider, and insurance claim data.

9. The personalized health management tool of claim 1, wherein the at least one medical condition comprises diabetes.

10. The personalized health management tool of claim 9, wherein the at least one medical condition farther comprises congestive heart failure.

11. The personalized health management tool of claim 1, wherein the information relating to the self-management of the at least one medical condition comprises tools for tracking patient progress.

12. The personalized health management tool of claim 1, further including a self-monitoring tool.

13. The personalized health management tool of claim 1, wherein the information relating to the at least one medical condition of a patient comprises at least one of background information relating to the at least one medical condition, treatments relating to the at least one medical condition, and information relating to daily living with the at least one medical condition.

14. A method for providing personalized health management information to one or more members of a health plan comprising:
identifying one or more members of a health plan having a specific medical condition;
gathering personalized health data for each identified member; and
providing each identified member with a personalized health management tool including information on the specific medical condition, information on self-management of the specific medical condition, and the personalized health data gathered for the respective identified member.

15. The method of claim 14, further comprising updating the personalized health management tool.

16. The method of claim 14, wherein the information relating to the specific medical condition comprises background information relating to the specific medical condition, treatments relating to the specific medical condition, information relating to daily living with the specific medical condition, evidence-based guidelines, and resource information relating to healthcare assistance, financial assistance, or social issues assistance.

17. The method of claim 14, wherein the specific medical condition is a chronic medical condition.

18. The method of claim 14, wherein the specific medical condition is diabetes.

19. The method of claim 14, wherein the specific medical condition comprises more than one chronic medical condition.

20. A method for providing personalized health management information to one or more patients comprising:
identifying one or more patients having a specific medical condition;
gathering personalized health data for each identified patient; and
providing each identified patient with a personalized health management tool including information on the specific medical condition, information on self-management of the specific medical condition, and the personalized health data gathered for the respective identified patient.
